(19) United States
(12) Patent Application Publication
(10) Pub. No.: US 2011/0237895 A1
(43) Pub. Date: Sep. 29, 2011

(54) IMAGE CAPTURING METHOD AND APPARATUS

(75) Inventors: Koji Yoshida, Ashigarakami-gun (JP); Hitoshi Shimizu, Ashigarakami-gun (JP); Kazuhiko Katakura, Ashigarakami-gun (JP); Takayuki Iida, Ashigarakami-gun (JP)

(73) Assignee: FUJIFILM Corporation, Tokyo (JP)

(21) Appl. No.: 12/979,272

(22) Filed: Dec. 27, 2010

(30) Foreign Application Priority Data
Mar. 25, 2010 (JP) 069501/2010

(51) Int. Cl. A61B 1/06

(52) U.S. Cl. 600/180

(57) ABSTRACT

An image capturing apparatus which includes a light projection unit for projecting light of a different wavelength range emitted from each of a plurality of light sources onto an observation area administered with a fluorescent agent, an imaging unit for receiving light emitted from the observation area irradiated with each light to capture an image corresponding to each light, and a light intensity ratio change unit for changing the light intensity ratio of light emitted from each of the light sources.

### BACKGROUND OF THE INVENTION

1. Field of the Invention

The present invention relates to an image capturing method and apparatus in which light in different wavelength ranges is emitted from of a plurality of light sources and each light is projected onto an observation area administered with a fluorescent agent to capture an image corresponding to each light.

2. Description of the Related Art

Endoscope systems for observing tissues of body cavities are widely known and an electronic endoscope system that captures an ordinary image of an observation area in a body cavity by projecting white light onto the observation area and displaying the captured ordinary image on a monitor screen is widely used.

Further, as one of such endoscope systems, a system that obtains a fluorescence image of a blood vessel or a lymphatic vessel by administering, for example, indocyanine green (ICG) into a body in advance and detecting fluorescence of ICG in the blood vessel or lymphatic vessel by projecting excitation light onto the observation area is known as described, for example, in U.S. Pat. No. 6,804,549 and Japanese Unexamined Patent Publication No. 2007-244746.

In the diagnosis by the endoscope system described above, tissue information in a surface layer or tissue information from a surface layer to a deep layer of a living tissue is an important observation target. For example, in the case of gastrointestinal cancer, tumor vessels appear in a surface layer of mucosa from an early stage, and more swelling, meandering, or increased blood vessel density is normally observed for the tumor vessels in comparison with blood vessels appearing on a surface layer. Therefore, the type of a tumor can be identified by closely examining the nature and appearance of the blood vessels.

When, for example, performing the observation of the blood vessel image using the ICG described above, it is possible to observe blood vessels located in a deep layer in a fluorescence image since the near infrared light used as the excitation light has high penetration into a living body, but the blood vessels located in a surface layer described above are blurred and can not be observed clearly.

In the mean time, it is proposed to obtain an image of a near surface area of a living tissue by directing narrow band light onto the observation area using a color filter as described, for example, in Japanese Patent No. 3583731. But, it is difficult to limit the transmission wavelength range of the color filter to a specific narrow band. In addition, the intensity of the narrow band light is insufficient, as the narrow band light is light transmitted through the color filter, causing a problem of degraded image quality. Further, the method described above has a similar problem when a deep layer blood vessel image is observed.

It is conceivable to obtain a surface layer blood vessel image by using a fluorescent agent that emits fluorescence in response to excitation light having a comparatively short wavelength. But, in this case, a deep layer blood vessel image can not be observed, although a surface layer blood vessel image can be observed.

Further, for example, when displaying a composite image by superimposing an ordinary image and a fluorescence image on top of each other, if the brightness of these images differs largely, the composite image becomes a very obscure image with an improper contrast.

The present invention has been developed in view of the circumstances described above, and it is an object of the present invention to provide an image capturing method and apparatus capable of, for example, emphatically displaying a blood vessel located at a desired depth from the body surface and displaying a composite image of an ordinary image and a fluorescence image superimposed on top of each other with an appropriate contrast.

### SUMMARY OF THE INVENTION

An image capturing method of the present invention is a method, including the steps of:

emitting light of a different wavelength range from each of a plurality of light sources, including at least one excitation light source;

projecting each light onto an observation area administered with a fluorescent agent; and

receiving light emitted from the observation area irradiated with each light and capturing an image corresponding to each light,

wherein the light intensity ratio of light emitted from each of the light sources is changed.

An image capturing apparatus of the present invention is an apparatus, including:

a light projection unit for projecting light of a different wavelength range emitted from each of a plurality of light sources, including at least one excitation light source, onto an observation area administered with a fluorescent agent;

an imaging unit for receiving light emitted from the observation area irradiated with each light and capturing an image corresponding to each light; and

a light intensity ratio change unit for changing the light intensity ratio of light emitted from each of the light sources.

In the image capturing apparatus of the present invention, the apparatus may include, as the light sources, a plurality of excitation light sources, and the light intensity ratio change unit may be a unit that changes the light intensity ratio of light emitted from each of the excitation light sources.

Further, the apparatus may include, as one of the light sources, an ordinary light source that emits ordinary light; and

the light intensity ratio change unit may be a unit that changes the light intensity ratio of the excitation light emitted from the excitation light source and the ordinary light emitted from the ordinary light source.

Still further, the excitation light emitted from each of the plurality of excitation light sources may be light that excites each of a plurality of corresponding fluorescent agents administered to the observation area.

Further, the light intensity ratio change unit may be a unit that changes the light intensity ratio such that the intensity of each image captured by the imaging unit through the projection of each excitation light is different.

Still further, the light intensity ratio change unit may be a unit that changes the light intensity ratio such that the intensity of each image captured by the imaging unit through the projection of each excitation light is identical.

Further, the imaging unit may be a unit that includes a plurality of image sensors, each for capturing an image corresponding to each light, and the light intensity ratio change unit may be a unit that changes the light intensity ratio of each light according to a value of light intensity ratio determined in advance based on a sensitivity of each of the image sensors.

Still further, the imaging unit may be a unit that includes a single image sensor for capturing an image corresponding to each light, and the light intensity ratio change unit may be a unit that changes the light intensity ratio of each light according to a value of light intensity ratio determined in advance based on a sensitivity of the image sensor.

According to the present invention, an image capturing method and apparatus is provided for emitting light of a different wavelength range from each of a plurality of light sources, including at least one excitation light source, projecting each light onto an observation area administered with a fluorescent agent, and receiving light emitted from the observation area irradiated with each light and capturing an image corresponding to each light in which the light intensity ratio of light emitted from each of the light sources is changed. This allows a blood vessel located at a desired depth from the body surface to be emphatically displayed by changing, for example, the light intensity ratio between light of a shorter wavelength range and light of a longer wavelength range. Further, by changing, for example, the light intensity ratio between the white light and excitation light, a composite image of an ordinary image and a fluorescence image superimposed on top of each other may be displayed with an appropriate contrast.

### BRIEF DESCRIPTION OF THE DRAWINGS

FIG. 1 is an overview of a rigid endoscope system that employs an embodiment of the image capturing apparatus of the present invention.

FIG. 2 is a schematic configuration diagram of the body cavity insertion section shown in FIG. 1.

FIG. 3 is a schematic view of a tip portion of the body cavity insertion section.

FIG. 4 is a cross-sectional view taken along the line 4-4′ in FIG. 3.

FIG. 5 illustrates a spectrum of light outputted from each light projection unit of the body cavity insertion section, and spectra of fluorescence and reflection light emitted/reflected from an observation area irradiated with the light.

FIG. 6 is a schematic configuration diagram of an imaging unit according to a first embodiment.

FIG. 7 illustrates spectral sensitivity of the imaging unit of the first embodiment.

FIG. 8 is a block diagram of a processor and a light source unit, illustrating schematic configurations thereof.

FIG. 9 is a block diagram of the image processing unit shown in FIG. 8, illustrating a schematic configuration thereof.

FIG. 10 is a schematic view illustrating blood vessels of surface and deep layers.

FIG. 11 is a schematic view for explaining a concept of a deep portion fluorescence image generation method.

FIGS. 12A to 12E illustrate timing charts of imaging timings of an ordinary image, an ICG fluorescence image, and a luciferase fluorescence image in the first embodiment of the image capturing apparatus of the present invention.

FIG. 13 is a flowchart for explaining an operation of the image capturing apparatus for displaying an ordinary image, a fluorescence image, and a composite image.

FIG. 14 is a flowchart for explaining line segment extraction using edge detection.

FIG. 15 is a schematic configuration diagram of an imaging unit of the image capturing apparatus according to a second embodiment.

FIG. 16 illustrates spectral sensitivity of the imaging unit of the second embodiment.

FIGS. 17A to 17C illustrate a method of imaging each image by a high sensitivity image sensor of imaging unit of the second embodiment.

FIGS. 18A to 18E illustrate timing charts of imaging timings of an ordinary image, an ICG fluorescence image, and a luciferase fluorescence image in the second embodiment of the image capturing apparatus of the present invention.

### DESCRIPTION OF THE PREFERRED EMBODIMENTS

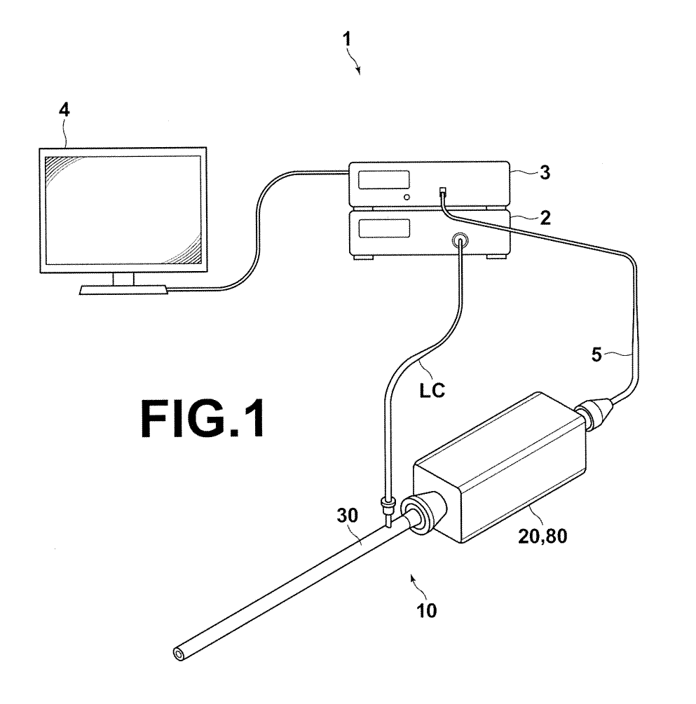

Hereinafter, a rigid endoscope system that employs a first embodiment of the image capturing method and apparatus of the present invention will be described in detail with reference to the accompanying drawings. FIG. 1 is an overview of rigid endoscope system 1 of the present embodiment, illustrating a schematic configuration thereof.

As shown in FIG. 1, rigid endoscope system 1 of the present embodiment includes light source unit 2 for emitting blue light, near infrared light, and near ultraviolet light, rigid endoscope imaging device 10 for guiding and directing the three types of light emitted from light source unit 2 to an observation area and capturing a fluorescence image based on fluorescence emitted from the observation area irradiated with excitation light and an ordinary image based on reflection light reflected from the observation area irradiated with white light, processor 3 for performing predetermined processing on image signals obtained by rigid endoscope imaging device 10 and controlling the intensity of each light emitted from light source unit 2, and monitor 4 for displaying the fluorescence and ordinary images of the observation area based on a display control signal generated in processor 3.

As shown in FIG. 1, rigid endoscope imaging device 10 includes body cavity insertion section 30 to be inserted into a body cavity, such as an abdominal cavity or a chest cavity, and imaging unit 20 for capturing an ordinary image and a florescence image of an observation area guided by the body cavity insertion section 30.

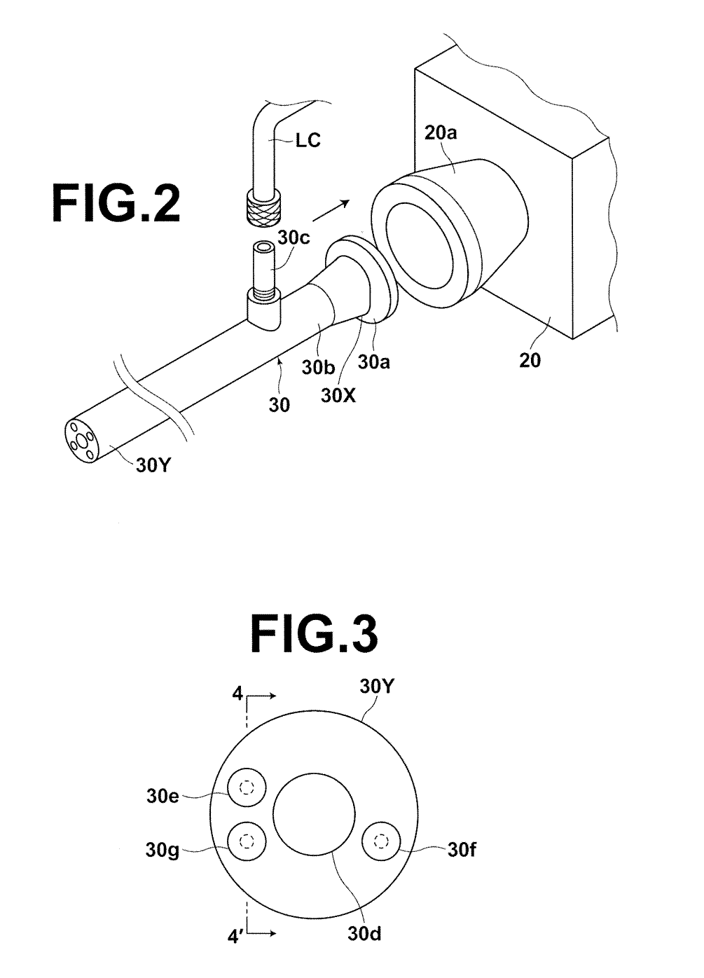

Body cavity insertion section 30 and imaging unit 20 are detachably connected, as shown in FIG. 2. Body cavity insertion section 30 includes connection member 30a, insertion member 30b, and cable connection port 30c.

Connection member 30a is provided at first end 30X of body cavity insertion section 30 (insertion member 30b), and imaging unit 20 and body cavity insertion section 30 are detachably connected by fitting connection member 30a into, for example, aperture 20a formed in imaging unit 20.

Insertion member 30b is a member to be inserted into a body cavity when imaging is performed in the body cavity. Insertion member 30b is formed of a rigid material and has, for example, a cylindrical shape with a diameter of about 5 mm. Insertion member 30b accommodates inside thereof a group of lenses for forming an image of an observation area, and an ordinary image and a fluorescence image of the observation area inputted from second end 30Y are inputted, through the group of lenses, to imaging unit 20 on the side of first end 30X.

Cable connection port 30c is provided on the side surface of insertion member 30b and an optical cable LC is mechanically connected to the port. This causes light source unit 2 and insertion member 30b to be optically linked through the optical cable LC.

As shown in FIG. 3, imaging lens 30d is provided in the approximate center of second end 30Y of body cavity insertion section 30 for forming an ordinary image and a fluorescence image, and white light projection lenses 30e and 30f for projecting white light are provide substantially symmetrically across the imaging lens 30d. The reason why two white light output lenses are provide symmetrically with respect to imaging lens 30d is to prevent a shadow from being formed in an ordinary image due to irregularity of the observation area.

Further, excitation light projection lens 30g for projecting near infrared light and near ultraviolet light, each of which is excitation light, onto the observation area at the same time is provided at second end 30Y of body cavity insertion section 30.

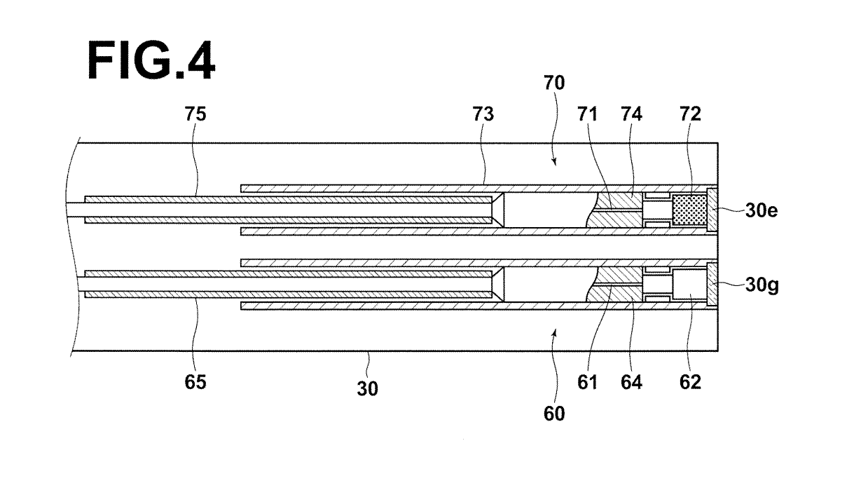

FIG. 4 is a cross-sectional view taken along the line 4-4′ in FIG. 3. As illustrated in FIG. 4, body cavity insertion section 30 includes inside thereof white light projection unit 70 and excitation light projection unit 60. White light projection unit 70 includes multimode optical fiber 71 for guiding blue light and fluorescent body 72 which is excited and emits visible light of green to yellow by absorbing a portion of the blue light guided through multimode optical fiber 71. Fluorescent body 72 is formed of a plurality of types of fluorescent materials, such as a YAG fluorescent material, BAM (BaMgAl₁₀O₁₇), and the like.

Tubular sleeve member 73 is provided so as to cover the periphery of fluorescent body 72, and ferrule 74 for holding multimode optical fiber 71 at the central axis is inserted in sleeve member 73. Further, flexible sleeve 75 is inserted between sleeve member 73 and multimode optical fiber 71 extending from the proximal side (opposite to the distal side) of ferrule 74 to cover the jacket of the fiber.

Excitation light projection unit 60 includes multimode optical fiber 61 for guiding the near infrared light and near ultraviolet light, and space 62 is provided between multimode optical fiber 61 and excitation light projection lens 30g. Also blue light projection unit 60 is provided with tubular sleeve member 63 covering the periphery of space 62, in addition to ferrule 64 and flexible sleeve 65, as in white light projection unit 70.

Note that the dotted circle in each projection lens in FIG. 3 represents the output end of the multimode optical fiber. As for the multimode optical fiber used in each light projection unit, for example, a thin optical fiber with a core diameter of 105 μm, a clad diameter of 125 μm, and an overall diameter, including a protective outer jacket, of 0.3 mm to 0.5 mm may be used.

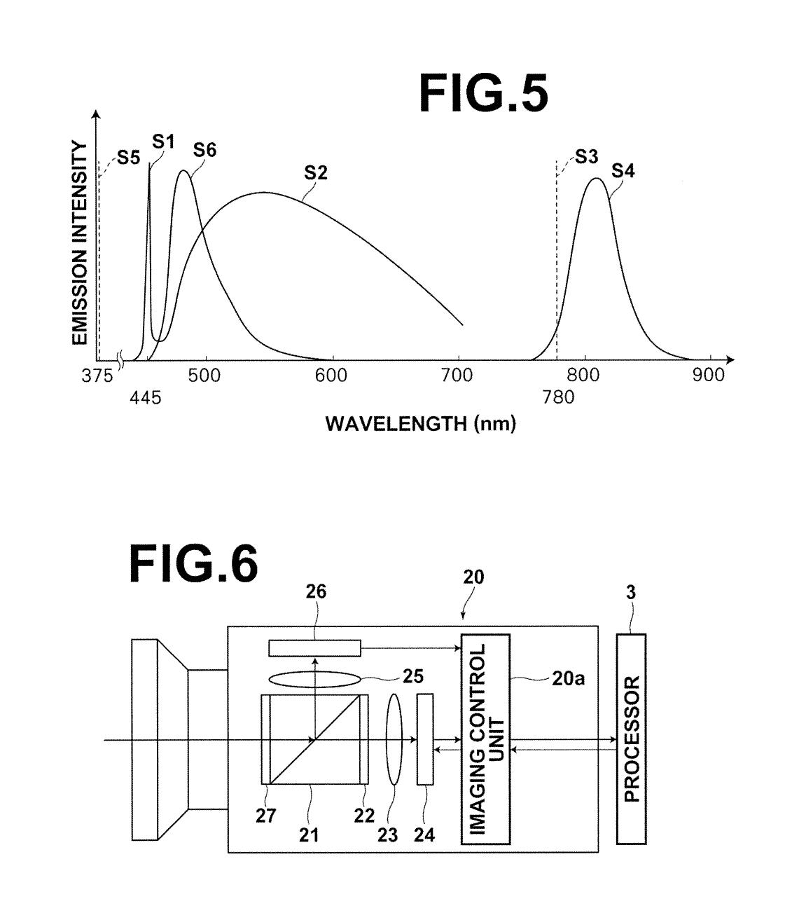

Each spectrum of light projected onto the observation area from each light projection unit, and spectra of fluorescence and reflection light emitted/reflected from the observation area irradiated with the projected light are shown in FIG. 5. FIG. 5 shows a blue light spectrum S1 projected through fluorescent body 72 of white light projection unit 70, a green to yellow visible light spectrum S2 excited and emitted from fluorescent body 72 of white light projection unit 70, a near infrared light spectrum S3 and a near ultraviolet light spectrum S5 projected from excitation light projection unit 60.

The term “white light” as used herein is not strictly limited to light having all wavelength components of visible light and may include any light as long as it includes light in a specific wavelength range, for example, primary light of R (red), G (green), or B (blue). Thus, in a broad sense, the white light may include, for example, light having wavelength components from green to red, light having wavelength components from blue to green, and the like. Although white light projection unit 70 projects the blue light spectrum S1 and visible light spectrum S2 shown in FIG. 5, the light of these spectra is also regarded as white light.

FIG. 5 further illustrates an ICG fluorescence spectrum S4 emitted from the observation area irradiated with the near infrared light spectrum S4 projected from excitation light projection unit 60 and a luciferase fluorescence spectrum S6 emitted from the observation area irradiated with the near ultraviolet light spectrum S5 projected from excitation light projection unit 60.

FIG. 6 shows a schematic configuration of imaging unit 20. Imaging unit 20 includes a first imaging system for generating an ICG fluorescence image signal of the observation area by imaging an ICG fluorescence image emitted from the observation area irradiated with the near infrared excitation light, and a second imaging system for generating a luciferase fluorescence image signal of the observation area by imaging a luciferase fluorescence image emitted from the observation area irradiated with the near ultraviolet light and generating an ordinary image signal of the observation area by imaging an ordinary image emitted from the observation area irradiated with the white light.

The first imaging system includes dichroic prism 21 that transmits the ICG fluorescence image emitted from the observation area, near infrared light cut filter 22 that transmits the ICG fluorescence image transmitted through dichroic prism 21 and cuts the near infrared excitation light transmitted through dichroic prism 21, first image forming optical system 23 that forms the ICG fluorescence image transmitted through near infrared light cut filter 22, and first high sensitivity image sensor 24 that captures the ICG fluorescence image formed by first image forming optical system 23.

The second imaging system includes dichroic prism 21 that reflects the ordinary image and luciferase fluorescence image reflected/emitted from the observation area, second image forming optical system 25 that forms the ordinary image and luciferase fluorescence image reflected by dichroic prism 21, and second high sensitivity image sensor 26 that captures the ordinary image and luciferase fluorescence image formed by the second image forming optical system 25 at different timings. Color filters of three primary colors, red (R), green (G), and blue (B) are arranged on the imaging surface of second high sensitivity image sensor 26 in a Beyer or a honeycomb pattern.

Further, violet light cut filter 27 is provided on the light incident surface of dichroic mirror 21 for cutting the entry of the near ultraviolet light. Violet light cut filter 27 is formed of a high-pass filter for cutting a 375 nm ultraviolet light wavelength range.

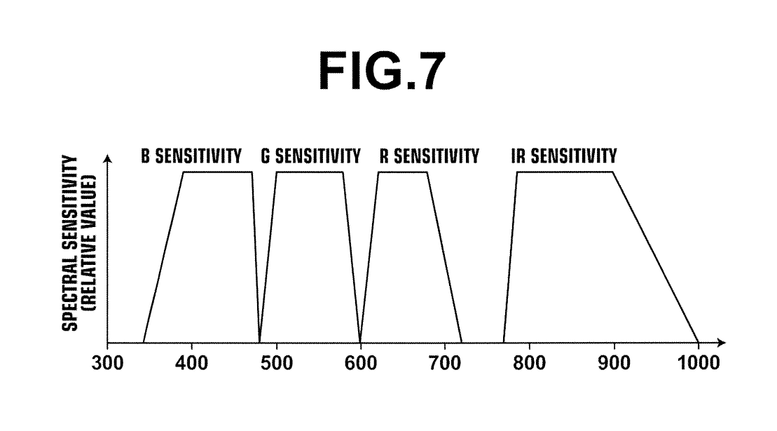

FIG. 7 is a graph of spectral sensitivity of imaging unit 20. More specifically, imaging unit 20 is configured such that the first imaging system has IR (near infrared) sensitivity, the second imaging system has R (red) sensitivity, G (green) sensitivity, and B (blue) sensitivity.

Imaging unit 20 further includes imaging control unit 20b. Imaging control unit 20b is a unit that controls high sensitivity image sensors 24, 26, performs CDS/AGC (correlated double sampling/automatic gain control) and A/D conversion on image signals outputted from high sensitivity image sensors 24, 26, and outputs the resultant image signals to processor 3 through cable 5 (FIG. 1).

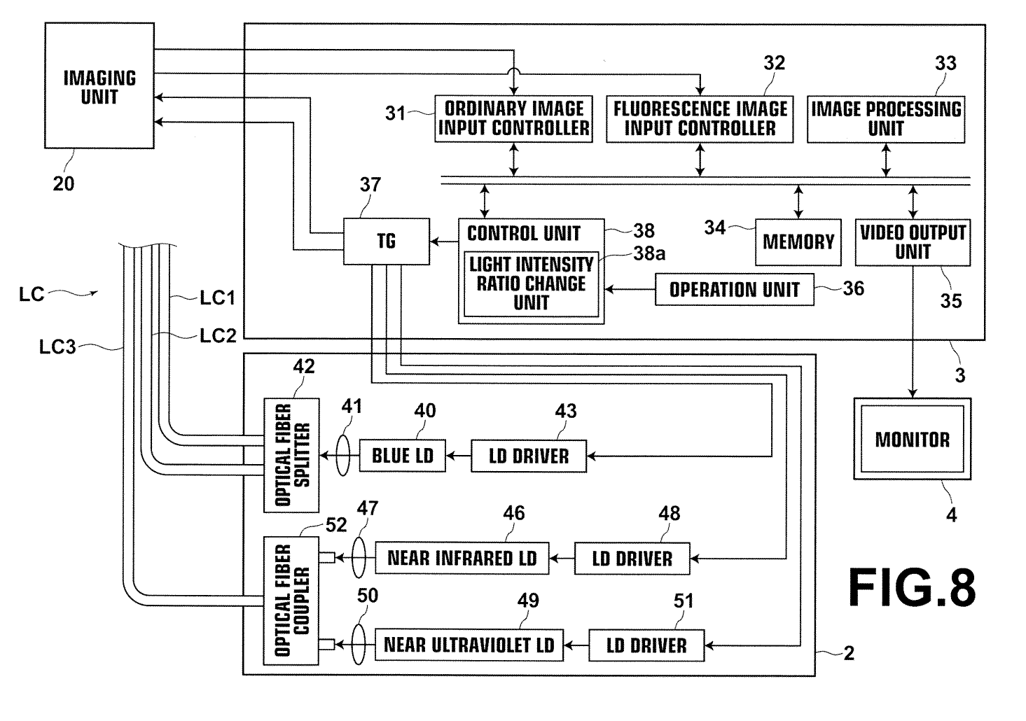

FIG. 8 is a block diagram of processor 3 and light source unit 2, illustrating internal structure thereof.

As shown in FIG. 8, processor 3 includes ordinary image input controller 31, fluorescence image input controller 32, image processing unit 33, memory 34, video output unit 35, operation unit 36, TG (timing generator) 37, and control unit 38.

Ordinary image input controller 31 and fluorescence image input controller 32 are each provided with a line buffer having a predetermined capacity and temporarily stores an ordinary image signal formed of image signals of RGB components with respect to one frame, or an ICG fluorescence image signal and a luciferase fluorescence image signal outputted from imaging control unit 20a of imaging unit 20. Then, the ordinary image signal stored in ordinary image input controller 31 and the fluorescence image signals stored in fluorescence image input controller 32 are stored in memory 34 via the bus.

Image processing unit 33 receives the ordinary image signal and fluorescence image signal for one frame read out from memory 34, performs predetermined processing on these image signals, and outputs the resultant image signals to the bus.

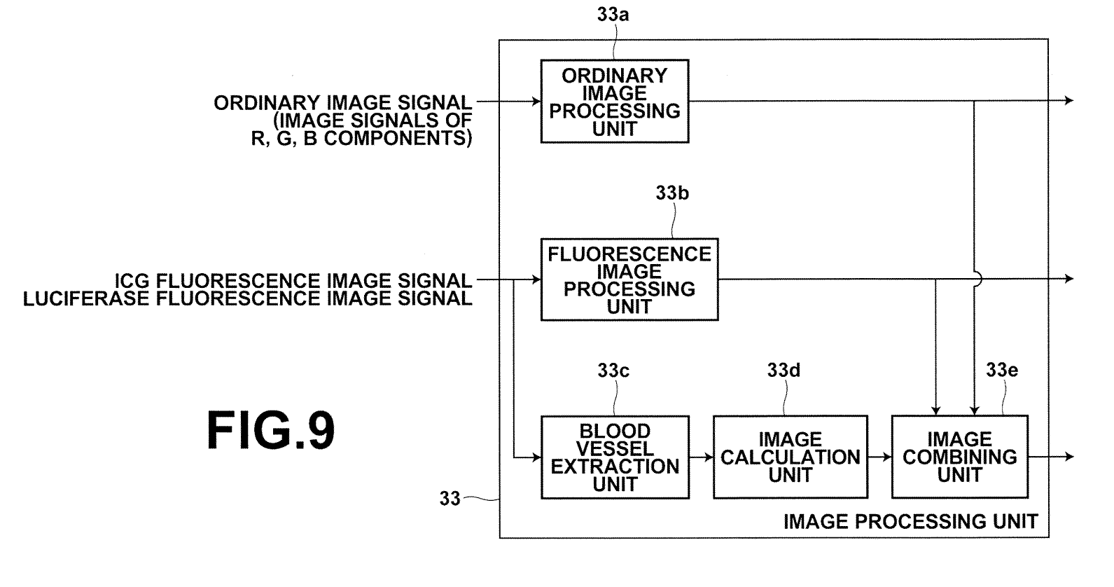

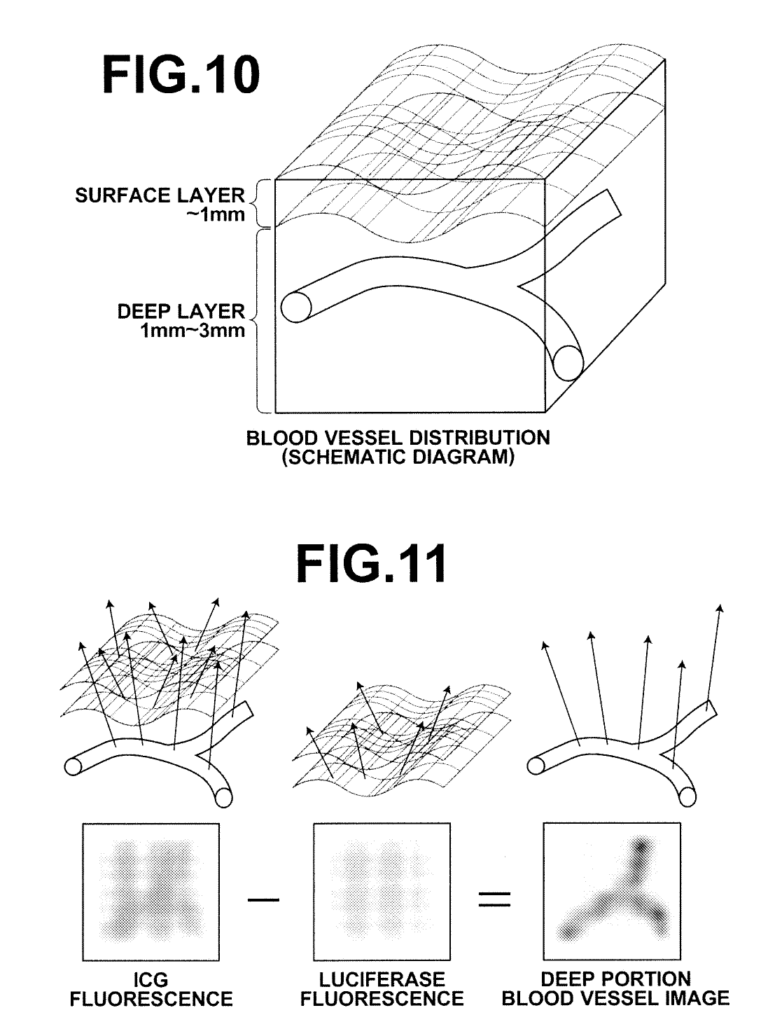

As shown in FIG. 9, image processing unit 33 includes ordinary image processing unit 33a that performs predetermined image processing, appropriate for an ordinary image, on an inputted ordinary image signal (image signals of RGB components) and outputs the resultant image signal, fluorescence image processing unit 33b that performs predetermined image processing, appropriate for a fluorescence image, on an inputted ICG fluorescence image signal and a luciferase fluorescence image signal and outputs the resultant image signals, and a blood vessel extraction unit 33c that extracts an image signal representing a blood vessel from the ICG fluorescence image signal and luciferase fluorescence image signal subjected to the image processing in fluorescence image processing unit 33b. Image processing unit 33 further includes image calculation unit 33d that subtracts an image signal representing a blood vessel extracted from the luciferase fluorescence image signal (hereinafter, “luciferase fluorescence blood vessel image signal”) from an image signal representing a blood vessel extracted from the ICG fluorescence image signal (hereinafter, “ICG fluorescence blood vessel image signal”) to generate a deep portion blood vessel image signal and image combining unit 33e that generates a combined image signal by combining the deep portion blood vessel image signal generated by image calculation unit 33d, the ICG fluorescence image signal, and the luciferase fluorescence image signal with the ordinary image signal outputted from ordinary image processing unit 33a. Processing performed by each unit of image processing unit 33 will be described in detail later.

Video output unit 35 receives the ordinary image signal, fluorescence image signal, and composite image signal outputted from image processing unit 33 via the bus, generates a display control signal by performing predetermine processing on the received signals, and outputs the display control signal to monitor 4.

Operation unit 36 receives input from the operator, such as various types of operation instructions and control parameters. TG 37 outputs drive pulse signals for driving high sensitivity image sensors 24 and 26 of imaging unit 20, and LD drivers 43, 48, and 51 of light source unit 2, to be described later.

Control unit 36 performs overall control of the system. In addition, control unit 36 includes light intensity ratio change unit 38a that changes the ratio of the intensities of blue light, near infrared light, and near ultraviolet light emitted from light source unit 2, to be described later. Changing of the light intensity ratio by light intensity ratio change unit 38a will be described in detail later.

As shown in FIG. 8, light source unit 2 includes blue LD light source 40 that emits blue light with a center wavelength of 445 nm, condenser lens 41 that condenses the blue light emitted from blue LD light source 40 and inputs the condensed blue light to optical fiber splitter 42, optical fiber splitter 42 that inputs the received blue light to optical cables LC1 and LC2 at the same time, and LD driver 43 that drives blue LD light source 40.

Light source unit 2 further includes near infrared LD light source 46 that emits 750 to 790 nm near infrared light, condenser lens 47 that condenses the near infrared light and inputs the condensed near infrared light to optical fiber coupler 52, and LD driver 48 that drives near infrared LD light source 46.

Light source unit 2 further includes near ultraviolet LD light source 49 that emits 300 to 450 nm near ultraviolet light, condenser lens 50 that condenses the near ultraviolet light and inputs the condensed near ultraviolet light to optical fiber coupler 52, and LD driver 51 that drives near ultraviolet LD light source 49.

Optical fiber coupler 52 is a device for inputting the near infrared light emitted from near infrared LD light source 46 and the near ultraviolet light emitted from near ultraviolet LD light source 49 to the input end of optical cable LC 3.

In the present embodiment, near infrared light and near ultraviolet light are used as the two types of excitation light, but excitation light having other wavelengths may also be used as the two types of excitation light as long as the wavelength of either one of them is shorter than that of the other and the excitation light is determined appropriately according to the type of fluorochrome administered to the observation area or the type of living tissue for causing autofluorescence.

Light source 2 is optically coupled to rigid endoscope imaging device 10 through optical cable LC, in which optical cables LC1, LC2 are optically coupled to multimode optical fibers 71 of white light projection unit 70 and optical cable LC3 is optically coupled to multimode optical fiber 61 of excitation light projection unit 60.

An operation of the rigid endoscope system of the first embodiment will now be described.

Before going into detailed description of the system operation, a blood vessel image to be obtained in the present embodiment will be described using a schematic drawing. In the present embodiment, a blood vessel image is obtained using an ICG fluorescence image and a luciferase fluorescence image.

Here, the near infrared light used as the excitation light for the ICG fluorescence image reaches comparatively a deep layer from the body surface so that the ICG fluorescence image may clearly shows a blood vessel located in a deep layer of 1 to 3 mm deep from the body surface but a blood vessel located in a surface layer from the body surface to about 1 mm deep is blurred. On the other hand, the ultraviolet light used as the excitation light for the luciferase fluorescence image has a shorter wavelength so that the luciferase fluorescence image can not show a deep layer blood vessel although a blood vessel located in a surface layer from the body surface to about 1 mm deep appears clearly.

Consequently, in the present embodiment, blood vessel images are displayed appropriately from the deep layer to the surface layer according to each depth range by changing the intensity ratio between the near infrared light and near ultraviolet light in view of the nature of the ICG fluorescence image and luciferase fluorescence image described above.

When only a deep portion blood vessel image is to be obtained, if only an ICG fluorescence image is obtained, the ICG fluorescence image includes not only the deep portion blood vessel image but also image information of a surface layer blood vessel located within a depth of 1 mm from the body surface, so that the surface layer blood vessel image appears as unnecessary information. On the other hand, the luciferase fluorescence image includes only image information of a surface blood vessel located in a surface layer as described above.

Therefore, when obtaining only a deep portion blood vessel image, the image is obtained by subtracting a luciferase fluorescence image from an ICG fluorescence image, as illustrated in FIG. 11. Here, in order for the subtraction to be performed appropriately, the intensity ratio between the near infrared light and near ultraviolet light is changed such that the magnitude of the ICG fluorescence image signal and the magnitude of the luciferase fluorescence image signal become identical.

Now, a specific operation of the rigid endoscope system of the present embodiment will be described.

First, body cavity insertion section 30 is inserted into a body cavity by the operator and the tip of body cavity insertion section 30 is placed adjacent to an observation area. Here, it is assumed that ICG and luciferase have already been administered to the observation area.

Here, an operation of the system for capturing an ordinary image will be described first. When capturing an ordinary image, blue light emitted from blue LD light source 40 of light source unit 2 is inputted to optical cables LC1 and LC2 through condenser lens 41 and optical fiber splitter 42. Then, the blue light is guided through optical cables LC1 and LC2 and inputted to body cavity insertion section 30, and further guided through multimode optical fibers 71 of white light projection unit 70 in body cavity insertion section 30. Thereafter, a certain portion of the blue light outputted from the output end of each multimode optical fiber 71 is transmitted through fluorescent body 72 and directed to the observation area, while the remaining portion of the blue light other than the certain portion is subjected to wavelength conversion to green to yellow visible light by fluorescent body 72 and directed to the observation area. That is, the observation area is irradiated with white light formed of the blue light and green to yellow visible light.

Then, an ordinary image based on reflection light reflected from the observation area irradiated with the white light is captured.

More specifically, the ordinary image is captured in the following manner. Reflection light reflected from the observation area irradiated with the white light is inputted to insertion member 30b from imaging lens 30d at the tip 30Y of insertion member 30b, then guided by the group of lenses inside of the insertion member 30b, and outputted to imaging unit 20.

The reflection light inputted to imaging unit 20 is transmitted through ultraviolet light cut filter 27, reflected in a right angle direction by dichroic prism 21, formed on the imaging surface of second high sensitivity image sensor 26 by second image forming optical system 25, and captured by second high sensitivity image sensor 26.

Then, R, G, and B image signals outputted from second high sensitivity image sensor 26 are respectively subjected to CDS/AGC (correlated double sampling/automatic gain control) and A/D conversion in imaging control unit 20a and outputted to processor 3 through cable 5.

Next, an operation of the system for capturing an ICG fluorescence image and a luciferase fluorescence image will be described.

When capturing an ICG fluorescence image and a luciferase fluorescence image, near infrared light emitted from near infrared LD light source 46 of light source unit 2 is inputted to optical fiber coupler 52 by condenser lens 47 and near ultraviolet light emitted from near ultraviolet LD light source 49 is inputted to optical fiber coupler 52 by condenser lens 50. Then, the near infrared light and near ultraviolet light are combined in the optical fiber coupler 52 and inputted to optical cable LC3.

The near infrared light and near ultraviolet light are inputted to body cavity insertion section 30 through optical cable LC3, then guided through multimode optical fiber 61 of excitation light projection unit 60 in body cavity insertion section 30, and projected onto the observation unit at the same time.

The ICG fluorescence image emitted from the observation area irradiated with the near infrared light and the luciferase fluorescence image emitted from the observation area irradiated with the near ultraviolet light are inputted to insertion member 30b from imaging lens 30d at the tip 30Y of insertion member 30b, then guided by the group of lenses inside of the insertion member 30b, and outputted to imaging unit 20.

The ICG fluorescence image inputted to imaging unit 20 is transmitted through ultraviolet light cut filter 27, dichroic prism 21, and near infrared light cut filter 22, formed on the imaging surface of first high sensitivity image sensor 24 by first image forming optical system 23, and captured by first high sensitivity image sensor 24. The ICG fluorescence image outputted from first high sensitivity image sensor 24 is subjected to CDS/AGC (correlated double sampling/automatic gain control) and A/D conversion in imaging control unit 20a and outputted to processor 3 through cable 5.

In the mean time, the luciferase fluorescence image is transmitted through ultraviolet light cut filter 27, reflected in a right angle direction by dichroic prism 21, formed on the imaging surface of second high sensitivity image sensor 26 by second image forming optical system 25, and captured by second high sensitivity image sensor 26.

Then, R, G, and B image signals outputted from second high sensitivity image sensor 26 are subjected to CDS/AGC (correlated double sampling/automatic gain control) and A/D conversion in imaging control unit 20a, and outputted to processor 3 through cable 5.

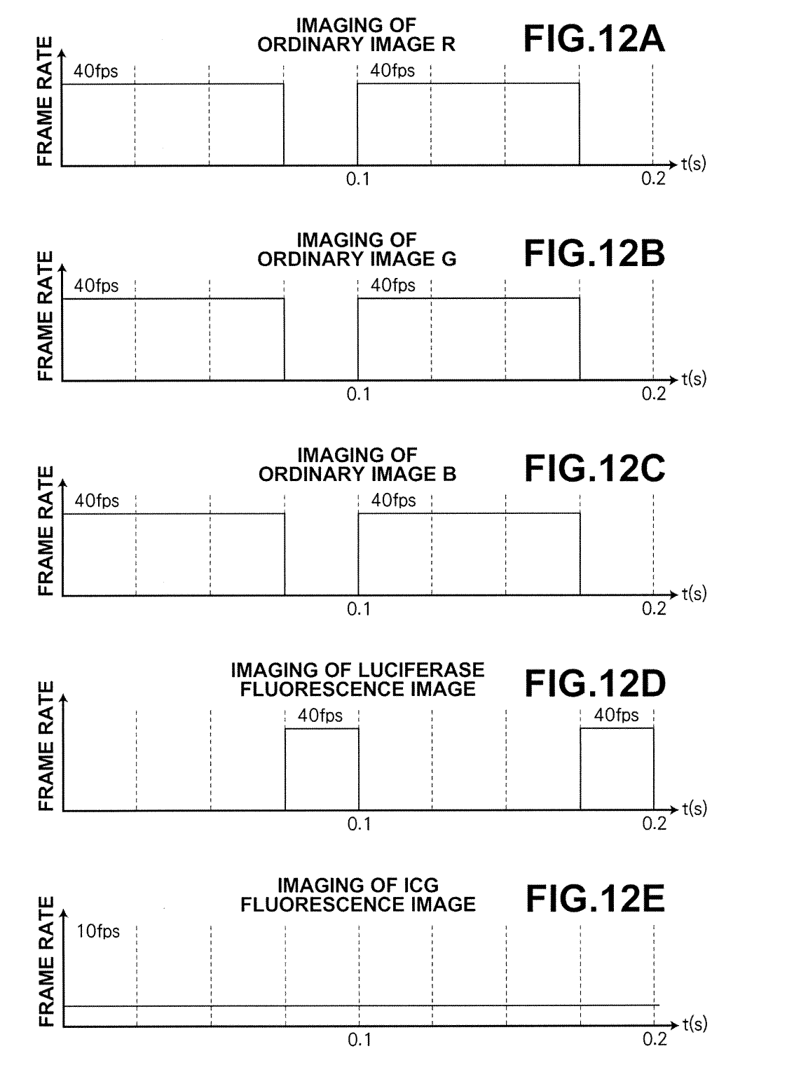

Now, referring to FIGS. 12A to 12E, there is provided timing charts of imaging timing of each of the ordinary image, ICG image, and luciferase fluorescence image described above. In each of the timing charts of FIGS. 12A to 12E, the horizontal axis represents the elapsed time and vertical axis represents the frame rate of the high sensitivity image sensor.

FIGS. 12A to 12C illustrate the imaging timings of second high sensitivity image sensor 26 for imaging R, G, and B ordinary image signals respectively, FIG. 12D illustrates the imaging timing of second high sensitivity image sensor 26 for imaging a luciferase fluorescence image, and FIG. 12E illustrates the imaging timing of first high sensitivity image sensor 24 for imaging an ICG fluorescence image.

In the timing charts of R, G, and B ordinary image signals shown in FIGS. 12A to 12C, the imaging is performed with a period of 0.1 s, a duty ratio of 0.75, and a frame rate of 40 fps. In the timing chart of luciferase fluorescence image shown in FIG. 12D, the imaging is performed with a period of 0.1 s, a duty ratio of 0.25, and a frame rate of 40 fps. In the timing chart of ICG fluorescence image shown in FIG. 12E, the imaging is performed with a duty ratio of 1 and a frame rate of 10 fps.

As the ordinary image and luciferase fluorescence image have the same B color component and can not be imaged at the same time, they are imaged at different timings as shown in FIGS. 12A to 12C and FIG. 12D.

Blue LD light source 40, near infrared LD light source 46, and near ultraviolet light source 49 of light source unit 2 are drive controlled according to the timing charts of FIGS. 12A to 12E. Here, in the present embodiment, the intensity ratio between the near infrared light and near ultraviolet light is changed so that blood vessel images are displayed appropriately from the deep layer to the surface layer according to each depth range, as described above.

Further, when obtaining only a deep portion blood vessel image, the intensity ratio between the near infrared light and near ultraviolet light is changed so that the ICG fluorescence image signal and luciferase fluorescence image signal become identical in magnitude.

More specifically, depth information for a blood vessel image desired to be observed is inputted by the operator from input unit 36, and the depth information is inputted to intensity ratio change unit 38a. Then, intensity ratio change unit 38a obtains an intensity ratio between the near infrared light and near ultraviolet light according to the inputted depth information for the blood vessel image and outputs a control signal to TG 37 according to the intensity ratio.

Note that the intensity ratio change unit 38a includes a table or the like in which intensity ratios according to depth information of blood vessel images are preset. For example, such an intensity ratio that causes the luciferase fluorescence image signal to become greater in magnitude than the ICG fluorescence image signal is set between the near infrared light and near ultraviolet light for surface layer depth information, while for deep layer depth information (including surface layer), such an intensity ratio that causes the ICG fluorescence image signal to become greater in magnitude than the luciferase fluorescence image signal is set between the near infrared light and near ultraviolet light. Further, for only deep layer depth information, such an intensity ratio that causes the ICG fluorescence image signal to become identical in magnitude to the luciferase fluorescence image signal is set between the near infrared light and near ultraviolet light. The intensity ratio may be set in a stepwise manner between the deep layer and the surface layer.

The intensity ratio between the near infrared light and near ultraviolet light is determined in view of emission intensity characteristics of the ICG and luciferase, the sensitivity and gain of first high sensitivity image sensor 24 that captures the ICG fluorescence image and of second high sensitivity image sensor 26 that captures the luciferase fluorescence image, and the like.

As for the initial intensity ratio between the near infrared light and near ultraviolet light, a value that causes the ICG fluorescence image signal to become identical in magnitude to the luciferase fluorescence image signal may be set.

The intensity ration between the near infrared light and near ultraviolet light can be changed by the operation as needed while observing a fluorescence image displayed on monitor 4.

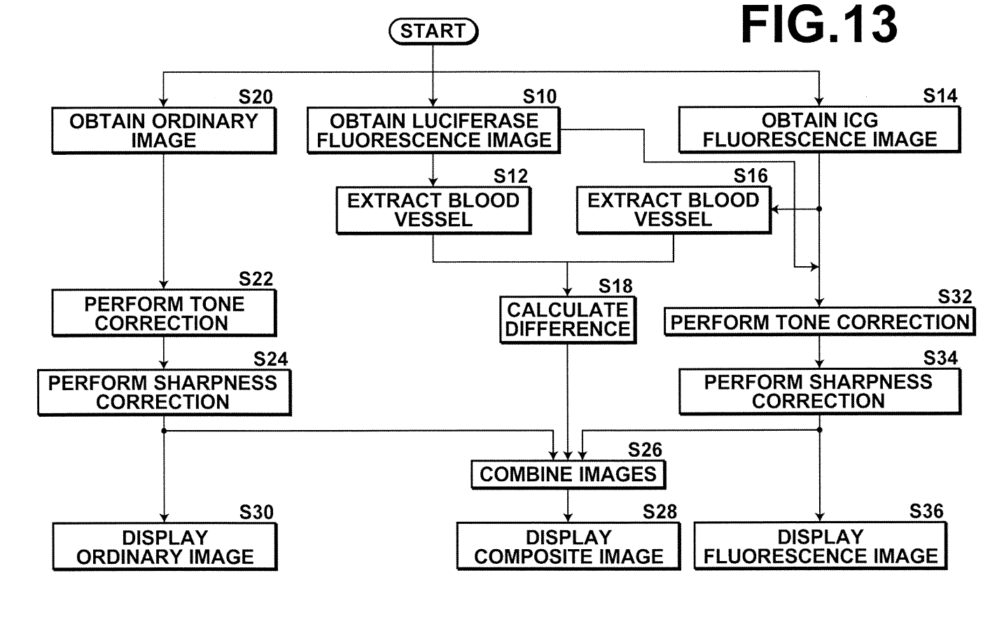

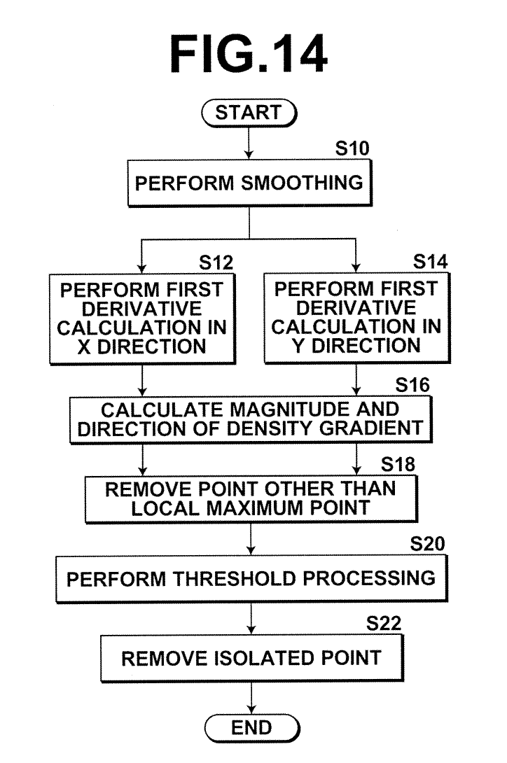

Next, an operation of the system for displaying an ordinary image, a fluorescence image, and a composite image based on the ordinary image signal formed of R, G, and B image signals, ICG fluorescence image signal, and luciferase fluorescence image signal obtained by imaging unit 20 will be described with reference to FIGS. 8, 9, and flowcharts shown in FIGS. 13, 14.

An operation for displaying an ordinary image, an ICG fluorescence image, and a luciferase fluorescence image will be described first.

The ordinary image signal formed of R, G, and B image signals inputted to processor 3 is temporarily stored in ordinary image input controller 31 and then stored in memory 34 (FIG. 13, S20). Ordinary image signals for one frame read out from memory 34 are subjected to tone correction and sharpness correction in ordinary image processing unit 33a of image processing unit 33 (FIG. 13, S22, S24), and outputted to video output unit 35.

Video output unit 35 generates a display control signal by performing predetermined processing on the inputted ordinary image signal and outputs the display control signal to monitor 4. Monitor 4 displays an ordinary image based on the inputted display control signal (FIG. 13, S30).

The ICG fluorescence image signal inputted to processor 3 is temporarily stored in fluorescence image input controller 32 and then stored in memory 34 (FIG. 13, S14) ICG fluorescence image signals for one frame read out from memory 34 are subjected to tone correction and sharpness correction in fluorescence image processing unit 33b of image processing unit 33 (FIG. 13, S32, S34), and outputted to video output unit 35.

Video output unit 35 generates a display control signal by performing predetermined processing on the inputted ICG fluorescence image signal and outputs the display control signal to monitor 4. Monitor 4 displays an ICG fluorescence image based on the inputted display control signal (FIG. 13, S36).

The luciferase fluorescence image signal inputted to processor 3 is temporarily stored in fluorescence image input controller 32 and then stored in memory 34 (FIG. 13, S14). Luciferase fluorescence image signals for one frame read out from memory 34 are subjected to tone correction and sharpness correction in fluorescence image processing unit 33b of image processing unit 33 (FIG. 13, S32, S34), and outputted to video output unit 35.

Video output unit 35 generates a display control signal by performing predetermined processing on the inputted luciferase fluorescence image signal and outputs the display control signal to monitor 4. Monitor 4 displays a luciferase fluorescence image based on the inputted display control signal (FIG. 13, S36).

Next, an operation of the system for displaying a composite image combining the ICG fluorescence image, luciferase fluorescence image, and ordinary image will be described.

When the composite image described above is generated, the ordinary image signal subjected to tone correction and sharpness correction in ordinary image processing unit 33a, and the ICG fluorescence image signal and luciferase fluorescence image signal subjected to tone correction and sharpness correction in fluorescence image processing unit 33b are inputted to image combining unit 33e.

Then, image combining unit 33e generates a composite image signal by combining the inputted ICG fluorescence image signal and luciferase fluorescence image signal with the ordinary image signal (FIG. 13, S26).

The composite image signal generated in image combining unit 33e is outputted to video output unit 35, and video output unit 35 generates a display control signal by performing predetermine processing on the received signal, and outputs the display control signal to monitor 4. Monitor 4 displays a composite image based on the inputted display control signal (FIG. 13, S28).

Next, an operation of the system for generating a deep portion blood vessel image based on an ICG fluorescence image signal and a luciferase fluorescence image signal, and displaying a composite image combining the deep portion blood vessel image with an ordinary image will be described.

An ICG fluorescence image signal and a luciferase fluorescence image signal inputted to processor 3 are temporarily stored in fluorescence image input controller 32 and then stored in memory 34 (FIG. 13, S10, S14).

The ICG fluorescence image signal and luciferase fluorescence image signal stored in memory 34 are inputted to blood vessel extraction unit 33c of image processing unit 33. Then, a blood vessel extraction is performed in blood vessel extraction unit 33c (FIG. 13, S12, S16).

The blood vessel extraction is implemented by performing line segment extraction. In the present embodiment, the line segment extraction is implemented by performing edge detection and removing an isolated point from the edge detected by the edge detection. Edge detection methods include, for example, Canny method using first derivation. A flowchart for explaining the line segment extraction using the Canny edge detection is shown in FIG. 14.

As shown in FIG. 14, filtering using a DOG (derivative of Gaussian) filter is performed on each of the ICG fluorescence image signal and luciferase fluorescence image signal (FIG. 14, S10 to S14). The filtering using the DOG filter is combined processing of Gaussian filtering (smoothing) for noise reduction with first derivative filtering in x, y directions for density gradient detection.

Thereafter, with respect to each of ICG fluorescence image signal and luciferase fluorescence image signal subjected to the filtering, the magnitude and direction of the density gradient are calculated (FIG. 14, S16). Then, a local maximum point is extracted and non-maxima other than the local maximum point are removed (FIG. 14, S18).

Then, the local maximum point is compared to a predetermined threshold value and a local maximum point with a value greater than or equal to the threshold value is detected as an edge (FIG. 14, S20). Further, an isolated point which is a local maximum point having a value greater than or equal to the threshold value but does not form a continuous edge is removed (FIG. 14, S22). The removal of the isolated point is processing for removing an isolated point not suitable as an edge from the detection result. More specifically, the isolated point is detected by checking the length of each detected edge.

The edge detection algorithm is not limited to that described above and the edge detection may also be performed using a LOG (Laplace of Gaussian) filter that combines Gaussian filtering for noise reduction with a Laplacian filter for edge extraction through secondary differentiation.

In the present embodiment, a blood vessel is extracted by line segment extraction using edge detection, but the method of blood vessel extraction is not limited to this and any method may be employed as long as it is designed for extracting a blood vessel portion, such as a method using hue or luminance.

With respect to each of the ICG fluorescence image signal and luciferase fluorescence image signal, an ICG fluorescence blood vessel image signal and a luciferase fluorescence blood vessel image signal are generated by extracting a blood vessel in the manner as described above. The luciferase fluorescence blood vessel image signal represents an image of a surface layer blood vessel located in a surface layer from the body surface of the observation area to a depth of 1 mm, while the ICG fluorescence blood vessel image signal includes both the surface layer blood vessel and a deep portion blood vessel located in a deep layer of a depth of 1 to 3 mm from the body surface.

Then, the ICG fluorescence blood vessel image signal and luciferase fluorescence blood vessel image signal generated in blood vessel extraction unit 33c are outputted to image calculation unit 33d and a deep portion blood vessel image is generated by subtracting the luciferase fluorescence blood vessel image signal from the ICG fluorescence blood vessel image signal in image calculation unit 33d (FIG. 13, S18).

Here, the depth information is changed to “only deep layer”, to change the intensity ratio between the near infrared light and near ultraviolet light such that the ICG fluorescence image signal and luciferase fluorescence image signal become identical in magnitude, as described above.

The deep portion blood vessel image signal generated in image calculation unit 33d in the manner as described above is outputted to image combining unit 33e. Image combining unit 33e also receives an ordinary image signal outputted from ordinary image processing unit 33a, and the deep portion blood vessel image is combined with the ordinary image signal, whereby a composite image signal is generated (FIG. 13, S26).

Then, the composite image signal generated in image combining unit 33e is outputted to video output unit 35.

Video output unit 35 generates a display control signal by performing predetermined processing on the inputted composite image signal outputs the display control signal to monitor 4. Monitor 4 displays a composite image based on the inputted display control signal (FIG. 13, S28).

Next, a rigid endoscope system that employs a second embodiment of the image capturing method and apparatus of the present invention will be described in detail. The overall schematic structure of the rigid endoscope system of the second embodiment is identical to that of the rigid endoscope system of the first embodiment shown in FIG. 1. Hereinafter, a description will be made focusing on a different between the first and second embodiments.

In imaging unit 20 of the rigid endoscope system of the first embodiment, two high sensitivity image sensors are used to capture an ordinary image, an ICG fluorescence image, and a luciferase fluorescence image, while in imaging unit 80 of the rigid endoscope system of the second embodiment, a single high sensitivity image sensor is used to capture an ordinary image, an ICG fluorescence image, and a luciferase fluorescence image.

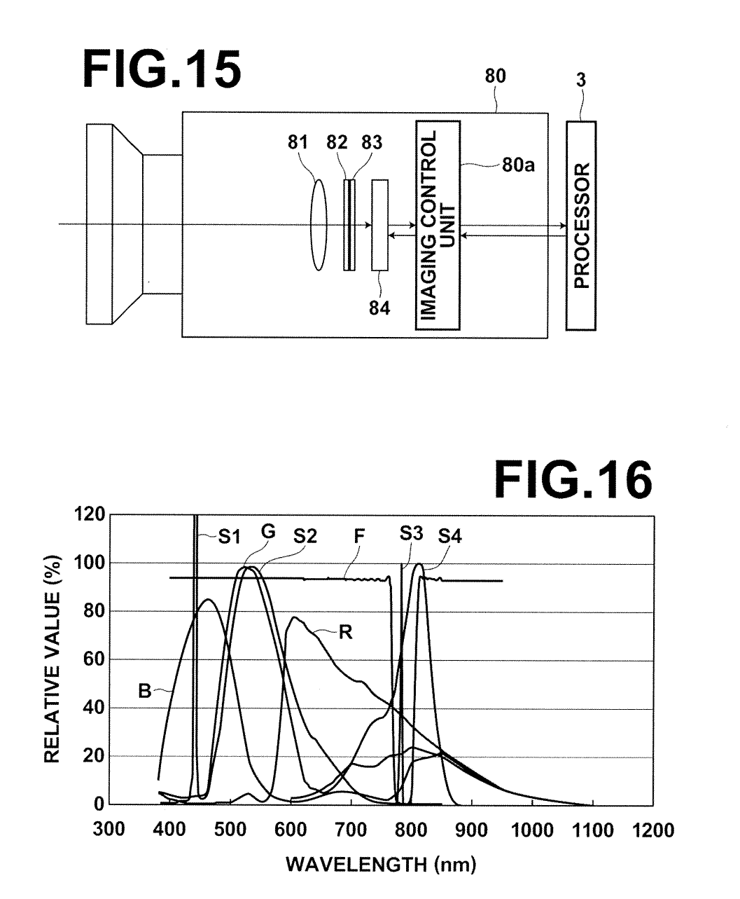

More specifically, as shown in FIG. 15, imaging unit 80 includes condenser lens 81 that condenses an ICG fluorescence image, a luciferase fluorescence image, and an ordinary image, ultraviolet light cut filter 82 that transmits the ICG fluorescence image, luciferase fluorescence image, and ordinary image condensed by condenser lens 81 and cuts ultraviolet light, infrared light cut filter 83 that transmits the ICG fluorescence image, luciferase fluorescence image, and ordinary image and cuts infrared light, and high sensitivity image sensor 84 that captures the ICG fluorescence image, luciferase fluorescence image, and ordinary image.

Ultraviolet light cut filter 82 is formed of a high-pass filter for cutting a 375 nm ultraviolet light wavelength range, as in the first embodiment.

Infrared light cut filter 83 is formed of a notch interference filter and has a filter characteristic of cutting off near infrared light and transmitting visible light and ICG fluorescence.

RGB color separation filters are arranged on the image surface of high sensitivity image sensor 84, as in second high sensitivity image sensor 26 of the first embodiment.

FIG. 16 illustrates spectral sensitivities R, G, and B of high sensitivity image sensor 84 of the present embodiment, white light spectra S1, S2 projected onto an observation area by white light projection unit 70, near infrared light spectrum S3, ICG fluorescence spectrum S4, and a filter characteristic F of near infrared light cut filter 83.

High sensitivity image sensor 84 of the present embodiment has sensitivity to the near infrared light region when the RBG color separation filters are not provided and each of RGB color separation filters have an identical transmittance in the near infrared light region, as shown in FIG. 16. Therefore, the ICG image is transmitted through each of R, G, and B filters and detected by image sensor 84.

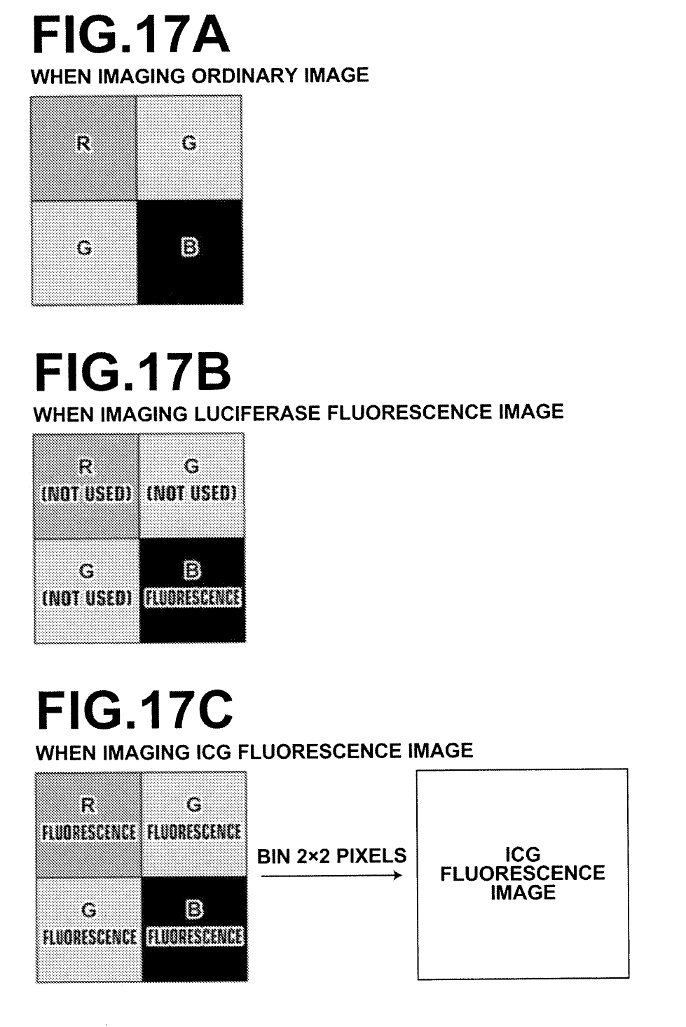

FIGS. 17A to 17C illustrate 2×2 pixels of high sensitivity image sensor 84. High sensitivity image sensor 84 is provided with R, G, B filters, as shown in FIGS. 17A to 17C. When capturing an ordinary image, an ordinary image transmitted through each of R, G, and B filters is detected, as shown in FIG. 17A, and an ordinary image signal is generated.

When capturing a luciferase fluorescence image, a luciferase fluorescence image transmitted through the B filter is detected as shown in FIG. 17B, and a luciferase fluorescence image signal is generated.

Further, when capturing an ICG fluorescence image, an ICG fluorescence image transmitted through each of R, G, and B filters is detected, as shown in FIG. 17C, and an ICG image signal is generated. Here, a signal of one pixel is generated by performing so-called binning reading of 2×2 pixels, however, considering that the transmittance of the filters is relatively low.

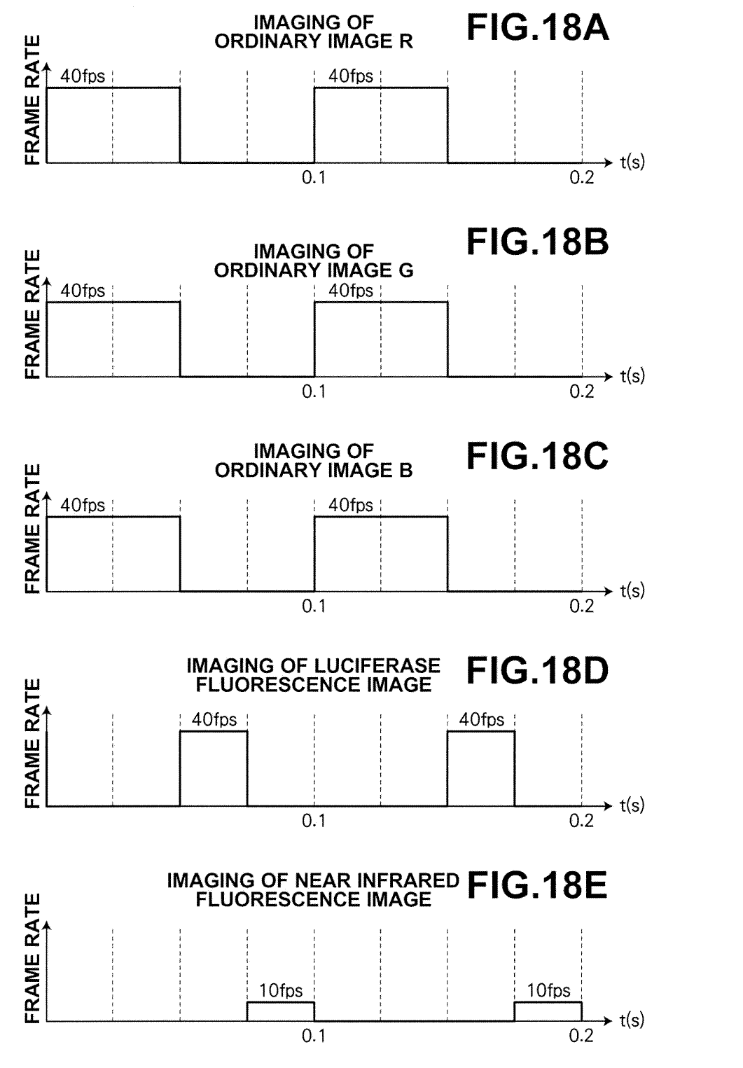

FIGS. 18A to 18E illustrate timing charts of imaging timing of each of ordinary image, ICG image, and luciferase fluorescence image in the present embodiment. In each of the timing charts of FIGS. 18A to 18E, the horizontal axis represents the elapsed time and vertical axis represents the frame rate of the high sensitivity image sensor.

FIGS. 18A to 18C illustrate the imaging timings of high sensitivity image sensor 84 for imaging R, G, and B ordinary image signals respectively, FIG. 18D illustrates the imaging timing of high sensitivity image sensor 84 for imaging a luciferase fluorescence image, and FIG. 18E illustrates the imaging timing of high sensitivity image sensor 84 for imaging an ICG fluorescence image.

In the timing charts of R, G, and B ordinary image signals shown in FIGS. 18A, to 18C, the imaging is performed with a period of 0.1 s, a duty ratio of 0.50, and a frame rate of 40 fps. In the timing chart of luciferase fluorescence image shown in FIG. 18D, the imaging is performed with a period of 0.1 s, a duty ratio of 0.25, and a frame rate of 40 fps. In the timing chart of ICG fluorescence image shown in FIG. 18E, the imaging is performed with a duty ratio of 0.25 and a frame rate of 10 fps.

In the present embodiment, the ordinary image, luciferase fluorescence image, and ICG fluorescence image are captured at different timings, as shown in FIGS. 18A to 18E.

With respect to the ICG fluorescence image, the frame rate is reduced to increase the charge storage time, considering a relatively low transmittance of the filters for the ICG fluorescence image.

In the second embodiment, blue LD light source 40, near infrared LD light source 46, and near ultraviolet light source 49 of light source unit 2 are drive controlled according to the timing charts of FIGS. 18A to 18E. Here, the intensity ratio between the near infrared light and near ultraviolet light is changed so that blood vessel images are displayed appropriately from the deep layer to the surface layer according to each depth range, as in the first embodiment.

Further, when obtaining only a deep portion blood vessel image, the intensity ratio between the near infrared light and near ultraviolet light is changed so that the ICG fluorescence image signal and luciferase fluorescence image signal become identical in magnitude.

The specific method of changing the intensity ratio between the near infrared light and near ultraviolet light is identical to that of the first embodiment. In the second embodiment, the intensity ratio between the near infrared light and near ultraviolet light is determined in view of emission intensity characteristics of the ICG and luciferase, the sensitivity and gain of high sensitivity image sensor 84, frame rate (charge storage time), and the like.

Other aspects and operation of the second embodiment are identical to those of the first embodiment.

In the first and second embodiments described above, the intensity ratio between the near infrared light and ultraviolet light is made changeable. But, for example, a configuration may be adopted in which the intensity of blue light emitted from blue LD light source 40 is also controlled and the intensity ratio between the near infrared light and/or near ultraviolet light and white light is changed.

More specifically, the light intensity ratio may be set such that the ICG fluorescence image and/or the luciferase fluorescence image and the ordinary image have substantially the same brightness, i.e., the ICG fluorescence image signal and/or the luciferase fluorescence image signal and the ordinary image signal become identical in magnitude.

In the first and second embodiments described above, a blood vessel image is displayed, but images representing other tube portions, such as lymphatic vessels, bile ducts, and the like may also be displayed.

Further, in the first and second embodiments described above, the fluorescence image capturing apparatus of the present invention is applied to a rigid endoscope system, but the apparatus of the present invention may also be applied to other endoscope systems having a soft endoscope. Still further, the fluorescence image capturing apparatus of the present invention is not limited to endoscope applications and may be applied to so-called video camera type medical image capturing systems without an insertion section to be inserted into a body cavity.

What is claimed is:

1. An image capturing method, comprising the steps of:
emitting light of a different wavelength range from each of a plurality of light sources, including at least one excitation light source;
projecting each light onto an observation area administered with a fluorescent agent; and
receiving light emitted from the observation area irradiated with each light and capturing an image corresponding to each light,
wherein the light intensity ratio of light emitted from each of the light sources is changed.

2. An image capturing apparatus, comprising:
a light projection unit for projecting light of a different wavelength range emitted from each of a plurality of light sources, including at least one excitation light source, onto an observation area administered with a fluorescent agent;
an imaging unit for receiving light emitted from the observation area irradiated with each light and capturing an image corresponding to each light; and
a light intensity ratio change unit for changing the light intensity ratio of light emitted from each of the light sources.

3. The image capturing apparatus of claim 2, wherein:
the apparatus comprises, as the light sources, a plurality of excitation light sources; and
the light intensity ratio change unit is a unit that changes the light intensity ratio of light emitted from each of the excitation light sources.

4. The image capturing apparatus of claim 2, wherein:
the apparatus comprises, as one of the light sources, an ordinary light source that emits ordinary light; and
the light intensity ratio change unit is a unit that changes the light intensity ratio of the excitation light emitted from the excitation light source and the ordinary light emitted from the ordinary light source.

5. The image capturing apparatus of claim 3, wherein the excitation light emitted from each of the plurality of excitation light sources is light that excites each of a plurality of corresponding fluorescent agents administered to the observation area.

6. The image capturing apparatus of claim 3, wherein the light intensity ratio change unit is a unit that changes the light intensity ratio such that the intensity of each image captured by the imaging unit through the projection of each excitation light is different.

7. The image capturing apparatus of claim 3, wherein the light intensity ratio change unit is a unit that changes the light intensity ratio such that the intensity of each image captured by the imaging unit through the projection of each excitation light is identical.

8. The image capturing apparatus of claim 2, wherein:
the imaging unit is a unit that includes a plurality of image sensors, each for capturing an image corresponding to each light; and
the light intensity ratio change unit is a unit that changes the light intensity ratio of each light according to a value of light intensity ratio determined in advance based on a sensitivity of each of the image sensors.

9. The image capturing apparatus of claim 2, wherein:
the imaging unit is a unit that includes a single image sensor for capturing an image corresponding to each light; and
the light intensity ratio change unit is a unit that changes the light intensity ratio of each light according to a value of light intensity ratio determined in advance based on a sensitivity of the image sensor.
